(19) United States
(12) Patent Application Publication
(10) Pub. No.: US 2024/0261442 A1
(43) Pub. Date: Aug. 8, 2024

(54) METHOD TO ASSESS NODAL DISEASE USING IRON OXIDE NANOPARTICLES IN MAGNETIC RESONANCE IMAGING

(71) Applicant: Imagion Biosystems, Inc., San Diego, CA (US)

(72) Inventors: Robert PROULX, Incline Village, CA (US); Yalia JAYALAKSHMI, Sunnyvale, CA (US); Marie ZHANG, San Diego, CA (US); Natalie YANG, Melbourne (AU); Wing Fai LAU, Melbourne (AU)

(73) Assignee: Imagion Biosystems, Inc., San Diego, CA (US)

(21) Appl. No.: 18/431,717

(22) Filed: Feb. 2, 2024

Related U.S. Application Data
(60) Provisional application No. 63/578,938, filed on Aug. 25, 2023.
(60) Provisional application No. 63/483,282, filed on Feb. 4, 2023.

(51) Int. Cl. A61K 49/18; A61B 5/055; B82Y 5/00

(52) U.S. Cl. CPC A61K 49/1875; A61B 5/055; A61K 49/1845; A61K 49/186; A61K 2123/00; B82Y 5/00

(57) ABSTRACT

This invention relates to a method to assess nodal disease by magnetic resonance imaging with a contrast agent. The contrast agent comprises biofunctional magnetic nanoparticles coated with polymers outside the iron core and conjugated with antibodies specific to the tumor type to be assessed. Images produced from this magnetic resonance imaging with a contrast agent show heterogenous hypo intensity or heterogenous architecture in regions where binding by the antibodies is present.

### BACKGROUND OF THE INVENTION

Magnetic resonance imaging (MRI) is a medical imaging technique that uses a magnetic field and computer-generated radio waves to create detailed images of the organs and tissues in your body. During magnetic resonance imaging, the magnetic field temporarily realigns water molecules in a mammalian body. Radio waves cause these aligned atoms to produce faint signals, which are used to create cross-sectional MRI images.

MRI with contrast or an imaging agent uses a contrast medium injected into a body for enhanced image quality. Such MRI contrast agents alter the relaxation times of nuclei within body tissues. The MRI scan with contrast highlights specific parts of the soft tissue by increasing the signal to noise, and is useful in identifying when there may be abnormalities within tissue, such as a tumor.

Contrast agents for MRI are detected indirectly through their ability to perturb water proton relaxation and modify MRI signal intensity. MR contrast agents primarily work by modifying the T1 (longitudinal or spin-lattice relaxation time), the T2 (spin-spin or transverse relaxation time) or the T2* (dephasing spin-spin) properties of the imaged tissue. The physical presence of SPIONs causes field perturbations in external magnetic fields which can then be indirectly detected by MRI. The SPIONs magnetic moments align with the MRI's magnetic field and become magnetized. When the field is removed the magnetic moments return to a random, non-magnetized state. This ability to go back and forth is called superparamagnetism.

SPIONs are dual contrast agents and modify both the T1 and T2/T2* tissue properties. SPIONs decrease the T1 relaxation properties of the imaged tissue, which generate positive MRI contrast on T1 weighted images. Gadolinium-based contrast agents (GBCAs) also generate contrast by decreasing T1 relaxation. SPIONs also cause a faster dephasing of spins of adjacent protons in tissues that have taken up the SPIONs and causes a reduction in the T2 and T2* MRI relaxivity rate. This T2/T2* reduction appears as a dark or hypointense contrast on T2 and T2* weighted images, resulting in negative MRI contrast images.

The most commonly used compounds for contrast enhancement are gadolinium-based. For large vessels such as the aorta and its branches, the dose can be as low as 0.1 mmol/kg of body mass. Higher concentrations are often used for finer vasculature. At much higher concentration, there is more T2 shortening effect of gadolinium, causing gadolinium brightness to be less than surrounding body tissues. However, at such concentration, it will cause greater toxicity to bodily tissues.

MRI has a wide range of applications in medical diagnosis and is an important diagnostic tool for cancer. When used without contrast, MRI provides images that are used in morphological assessments of possible tumors. With contrast, MRI provides images that are easier to assess for presence of abnormalities in the tissues.

After a new breast cancer diagnosis, nodal staging, which involves a combination of clinical assessment and radiographic imaging, is performed. Precise nodal staging is an essential component in the management of patients with breast cancer, as treatments depend on patient specific characteristics of the primary tumor, nodal status, and evaluation for distant metastatic disease (NCCN guidelines version 4, 2022). Although regional nodal assessment is crucial, there are variable practice patterns and imaging modalities employed based on available resources and institutional experience. Furthermore, regardless of negative imaging or radiographically concerning findings, pathologic confirmation through either needle biopsy or through operative extirpation of the sentinel lymph node (SLN) remains the gold standard.

The two most commonly employed radiographic modalities for assessing nodal metastasis are axillary ultrasound and Gd enhanced Magnetic Resonance Imaging (MRI), and the pooled diagnostic sensitivity and specificity of MRI is 75% to 80% and 89% to 91%, respectively, versus that of ultrasound, which is 49% to 87%, and 55% to 97%, respectively (Beenken 2003, Choi 2017). Given the significance of accurate nodal assessment, radiographic modalities still represent opportunities for improvement. Although ultrasound has the advantages of convenience, patient comfort, and decreased cost compared to MRI, its utility depends on the experience of the operator, which accounts for the wide variability in sensitivity and specificity. Ultrasound is also limited in its ability to scan for the extent of the locoregional disease burden (Cody 2012, Saksena 2021). On the other hand, MRI is less dependent on operator experience and can scan the entire nodal region, but findings suggestive of regional lymph node disease are not specific to the tumor and are only surrogates for possible tumor involvement such as size and morphologic changes. While the absence of enlarged regional lymph node disease can be assuring, the presence of abnormal nodes requires additional evaluation with an ultrasound and percutaneous biopsy.

Accurate nodal assessment has become even more important since the 2011 publication of the landmark American College of Surgeons Oncology Group (ACOSOG) Z0011 trial (Giulianio 2011, Giulianio 2017). This trial began the dramatic shift towards de-escalation of axillary surgery as the authors demonstrated that even with positive sentinel lymph nodes, women undergoing breast conservation therapy who met inclusion criteria can omit axillary lymph node dissection (ALND). Since the publication of these and other data, there continues to be growing interest in omitting axillary surgery and the sentinel lymph node biopsy (SLNB) procedure (Reiner 2018).

Under these changing circumstances, there is even more need for improved accuracy of noninvasive imaging methods (Jatoi 2021, Leenders 2019). A noninvasive and molecularly targeted, tumor-specific approach that serves as contrast enhancement to the well accepted imaging modality, such as MRI, will add value to existing ways of nodal staging as well as contribute to the evolving practice of surgical de-escalation and subsequent clinical decision making.

The present disclosure addresses these and other needs in the art.

### SUMMARY OF THE INVENTION

The present disclosure relates to reagents and methods involving the use of an imaging agent to assess target tissues, in particular axillary nodes, for health conditions such as tumors, in particular identify cancerous (e.g., HER2 positive) cells or tissue in axilla nodes. Exemplary methods are conducted by magnetic resonance imaging utilizing a superparamagnetic iron oxide nanoparticle (i.e., biofunctional magnetic nanoparticle) solution imaging agent.

According to certain embodiments, a method to assess target tissues by magnetic resonance imaging is provided, the method comprising: introducing a biofunctional magnetic nanoparticle solution into a subject, wherein the biofunctional magnetic nanoparticle solution is comprised of a plurality of biofunctional magnetic nanoparticles, each nanoparticle comprising an iron core coated with a layer of organic coating, one or more lipid or polymer, one or more targeting ligand adapted to bind a target molecule, and at least one stealth generating compound bound to the polymer coating layer; permitting biofunctional magnetic nanoparticle solution to bind the target molecule, if present, wherein the target molecule is indicative of a health condition in the subject; performing magnetic resonance imaging of the subject to obtain a magnetic resonance image or image file representative of a target tissue of the subject; assessing the magnetic resonance image or image file for an area of heterogenous hypointensity in the target tissue representative of the presence of biofunctional magnetic nanoparticles bound to the target molecule, whereby identifying the area of heterogenous hypointensity in the target tissue is indicative of the presence of the target molecule in the subject tissue. Often the methods further include subjecting the subject to treatment for the health condition, which treatment may involve administration of medicaments, biopsy, surgery, or foregoing biopsy or surgery. In certain embodiments, the polymer comprises poly(maleic anhydride-alt-octadecene). In often included embodiments, the polymer layer is functionalized with carboxylate groups or other polymers. In certain embodiments, the polymer layer is functionalized with a polyethylene glycol polymer. In often embodiments, the targeting ligand is conjugated to the nanoparticle structure by binding with the polymer layer, such as a layer of poly(maleic anhydride-alt-octadecene). Often the target ligand is capable of specifically binding a protein or a cell associated with a cancer. Frequently the cancer is breast cancer. In certain embodiments the target molecule is a HER2 protein, or domain or region thereof.

According to frequently included embodiments, identification of heterogenous hypointensity in the target tissue is further indicative of malignancy of the subject tissue.

Also according to certain embodiments, magnetic resonance imaging of the subject occurs within 8 hours to 72 after the biofunctional magnetic nanoparticle solution is introduced to the subject. In certain related embodiments, the magnetic resonance imaging of the subject occurs within 8 hours to 24 after the biofunctional magnetic nanoparticle solution is introduced to the subject. In certain related embodiments, the magnetic resonance imaging of the subject occurs within 24 hours to 36 after the biofunctional magnetic nanoparticle solution is introduced to the subject. In certain related embodiments, the magnetic resonance imaging of the subject occurs within 24 hours to 48 after the biofunctional magnetic nanoparticle solution is introduced to the subject. In certain related embodiments, the magnetic resonance imaging of the subject occurs within 24 hours to 72 after the biofunctional magnetic nanoparticle solution is introduced to the subject. In certain related embodiments, the magnetic resonance imaging of the subject occurs within 36 hours to 72 after the biofunctional magnetic nanoparticle solution is introduced to the subject. In certain related embodiments, the magnetic resonance imaging of the subject occurs within 48 hours to 72 after the biofunctional magnetic nanoparticle solution is introduced to the subject. In certain related embodiments, the magnetic resonance imaging of the subject occurs within 1 hour to 8 hours after the biofunctional magnetic nanoparticle solution is introduced to the subject. In certain related embodiments, the magnetic resonance imaging of the subject occurs within 72 hours to 168 hours after the biofunctional magnetic nanoparticle solution is introduced to the subject.

According to certain embodiments, a method to assess nodal disease by magnetic resonance imaging is provided, the method comprising: introducing a biofunctional magnetic nanoparticle solution via parenteral administration, including intravenous, intraperitoneal, peritumoral, subcutaneous, or intramuscular, for example, into a subareolar or peritumor region of a human subject diagnosed with or suspected of having breast cancer (e.g., HER-2 positive breast cancer); performing magnetic resonance imaging of the region of interest of the human subject, the axilla region comprising at least one node; assessing an image of the at least one node for heterogenous hypo-intensity; and assessing nodal disease of the at least one node, wherein the biofunctional magnetic nanoparticle solution comprises nanoparticle structures, each structure comprising an iron core surrounded with a layer of organic coating, a polymer layer, at least one stealth generating compound bound to the polymer coating layer, and a targeting ligand adapted to bind a target molecule indicative of the nodal disease. In certain embodiments, the polymer comprises poly(maleic anhydride-alt-octadecene). Also in certain embodiments, the targeting ligand is conjugated to the nanoparticle structure at the polymer coating layer. Often the target molecule is capable of specifically binding a protein or cell associated with a cancer. Frequently the cancer is breast cancer. In certain embodiments the target molecule is an HER2 protein, or another protein differentially expressed on cancer cells, or domain or region thereof. Also in frequent embodiments the targeting ligand comprises an anti-HER2 protein such as trastuzumab or a functional portion or binding domain thereof.

According to frequently included embodiments, identification of heterogenous hypointensity in the lymph node is further indicative of malignancy of the subject tissue.

According to certain embodiments, a method to evaluate target tissue by magnetic resonance imaging is provided, the method comprising: introducing a biofunctional magnetic nanoparticle solution into a subject, wherein the biofunctional magnetic nanoparticle solution is comprised of a plurality of biofunctional magnetic nanoparticles, each nanoparticle comprising an iron core coated with oleic acid, one or more lipid or polymer, one or more stealth generating compound, and one or more targeting ligand adapted to bind a target molecule; permitting the biofunctional magnetic nanoparticle solution to bind the target molecule, if present, wherein the target molecule is indicative of a health condition in the subject; permitting the biofunctional magnetic nanoparticle solution to bind the target molecule, if present, wherein the target molecule is indicative of a health condition in the subject; and assessing the magnetic resonance image or image file for an area of heterogenous hypointensity in the target tissue representative of the presence of biofunctional magnetic nanoparticles bound to the target molecule, whereby identifying the area of heterogenous hypointensity in the target tissue is indicative of the presence of the target molecule in the subject tissue. In certain embodiments, the polymer comprises poly(maleic anhydride-alt-octadecene). Also in certain embodiments, the targeting ligand is conjugated to the nanoparticle structure at the polymer layer. Often the target ligand is capable of specifically binding a protein or cell associated with a cancer. Frequently the cancer is breast cancer. In certain embodiments the target molecule is a HER2 protein, or domain or region thereof. Also in frequent embodiments the targeting ligand comprises an anti-HER2 antibody such as trastuzumab or a functional portion or binding domain thereof.

According to often included embodiments, a method to evaluate target tissue by magnetic resonance imaging is provided as above, wherein each of the nanoparticle structures within the nanoparticle solution has a diameter of between 10 and 150 nanometers. Also according to frequent embodiments, within any specific solution the nanoparticles have a poly dispersive index (PDI)<0.2. Also according to certain embodiments, within any specific solution the diameter of each of the nanoparticles in the nanoparticle solution often is uniform or relatively uniform. Also according to certain embodiments, within any specific solution the diameter of each of the nanoparticles in the nanoparticle solution often is between 55 and 90 nanometers.

Frequently according to other embodiments, a method to assess axillary nodal diseases by magnetic resonance imaging is provided as above, wherein the biofunctional magnetic nanoparticle solution is an aqueous nanoparticle solution in 0.9% NaCl containing 0.05% polysorbate 20. In certain embodiments, the solution comprises an isotonic solution that supports product stability and safe human injection.

Often according to embodiments, a method to evaluate target tissue by magnetic resonance imaging is provided as above, wherein the polyethylene glycol (PEG) polymers are methoxy polyethylene glycol 2000 and methoxy polyethylene glycol 10000. In certain embodiments, the PEG polymer is within a size range of between about 500 Da to at or about 20,000 Da.

According to other embodiments, a method to evaluate target tissue by magnetic resonance imaging is provided as above, wherein the organic coating layer is any organic acid, e.g., oleic acid.

Often according to embodiments, a method to evaluate target tissue by magnetic resonance imaging is provided as above, wherein polymer coating comprises dextran.

According to frequent embodiments, a method to evaluate target tissue diseases by magnetic resonance imaging is provided as above, wherein the antibody is an antibody or functional binding fragment thereof capable of specifically binding a HER2 protein such as trastuzumab or a functional binding fragment thereof.

Often according to embodiments, a method to evaluate target tissue by magnetic resonance imaging is provided as above, wherein the polymer coating outside the organic coating is poly(maleic anhydride-alt-octadecene) (POMA). In certain embodiments the POMA is functionalized with a carboxylate group. In such embodiments, the functionalized POMA is adapted to form a covalent bond with amine group on other materials such as protein (contain lysine which has an amine group) via EDC chemistry. While POMA is a frequently preferred polymer, other polymers may be included.

According to frequently included embodiments, a method to evaluate target tissue by magnetic resonance imaging is provided as above, wherein one lymph node is assessed.

Frequently according to other embodiments, a method to evaluate target tissue by magnetic resonance imaging is provided as above, wherein more than one lymph node is assessed.

Frequently according to other embodiments, a method to evaluate target tissue by magnetic resonance imaging is provided as above, wherein the performing magnetic resonance imaging uses a T1 imaging sequence as described herein.

Often according to embodiments, a method to evaluate target tissue by magnetic resonance imaging is provided as above, wherein the performing magnetic resonance imaging uses a T2 imaging sequence as described herein.

According to frequently included embodiments, a method to evaluate target tissue by magnetic resonance imaging is provided as above, wherein the human subject holds their breath while magnetic resonance imaging of the axilla region is conducted.

Frequently according to embodiments, a method to evaluate target tissue by magnetic resonance imaging is provided as above, wherein the biofunctional magnetic nanoparticle solution is injected into a peritumoral region of a tumor present on the human subject.

According to often embodiments, a method to evaluate target tissue by magnetic resonance imaging is provided as above, wherein the performing of magnetic resonance imaging is conducted using a 1.5 T or 3 T clinical scanner.

Frequently according to embodiments, a method to evaluate target tissue by magnetic resonance imaging is provided as above, further comprising assessing the image of the at least one lymph node for heterogenous architecture.

According to frequent embodiments, a method to evaluate target tissue by magnetic resonance imaging is provided as above, further comprising assessing the morphology of the at least one lymph node for presence of suspicious tumors.

Frequently according to other embodiments, a method to evaluate target tissue by magnetic resonance imaging is provided as above, further comprising performing a biopsy on the lymph node if assessed to be diseased.

Often according to embodiments, a method to evaluate target tissue by magnetic resonance imaging is provided as above, further comprising performing magnetic resonance imaging on the human subject 24 hours after the first imaging and reassessing the target tissue. In certain embodiments, magnetic resonance imaging on the human subject in one hour after dosing, 1 day after dosing, 2 days after dosing, 3 days after dosing, 4 days after dosing, 5 days after dosing, 6 days after dosing, or in a range of 1-3 days after dosing or 1-7 days after dosing.

Frequently according to embodiments, a method to evaluate target tissue by magnetic resonance imaging is provided as above, wherein the target molecule is capable of specifically binding a protein or cell associated with a cancer.

According to frequent embodiments, a method to evaluate target tissue by magnetic resonance imaging is provided as above, wherein the target molecule is a HER2 protein, or domain or region thereof.

Frequently according to embodiments, a method to evaluate target tissue by magnetic resonance imaging is provided as above, further comprising assessing the image of the target tissue for heterogenous architecture.

Often according to embodiments, a method to evaluate target tissue by magnetic resonance imaging is provided as above, further comprising subjecting the subject to treatment for the health condition, which treatment involves administration of medicaments, biopsy, surgery, or foregoing biopsy or surgery.

According to frequent embodiments, a method to evaluate target tissue by magnetic resonance imaging is provided as above, wherein identification of heterogenous hypointensity in the tissue is further indicative of malignancy of the subject tissue.

Often according to embodiments, a method to evaluate target tissue by magnetic resonance imaging is provided as above, wherein identification of heterogenous architecture in the target tissue is further indicative of malignancy of the subject tissue.

Also included herein are medicaments for use in magnetic resonance imaging formulated for use in evaluating and/or treating a health condition characterized by the presence a protein or cell associated with a cancer, the methods comprising: introducing a biofunctional magnetic nanoparticle solution into a subject, wherein the biofunctional magnetic nanoparticle solution is comprised of a plurality of biofunctional magnetic nanoparticles, each nanoparticle comprising an iron core coated with oleic acid, one or more lipid or polymer, and one or more targeting ligand adapted to bind a target molecule; permitting biofunctional magnetic nanoparticle solution to bind the target molecule, if present, wherein the target molecule is indicative of a health condition in the subject; performing magnetic resonance imaging of the subject to obtain a magnetic resonance image or image file representative of a target tissue of the subject; assessing the magnetic resonance image or image file for an area of heterogenous hypointensity in the target tissue representative of the presence of biofunctional magnetic nanoparticles bound to the target molecule, whereby identifying the area of heterogenous hypointensity in the target tissue is indicative of the presence of the target molecule in the subject tissue. Often the methods further include subjecting the subject to treatment for the health condition, which treatment may involve administration of medicaments, biopsy, surgery, or foregoing biopsy or surgery. In certain embodiments, the polymer comprises poly(maleic anhydride-alt-octadecene). Also in certain embodiments, the targeting ligand is conjugated to the nanoparticle structure at the polymer coating layer. Often the target molecule is capable of specifically binding a protein or cell associated with a cancer. Frequently the cancer is breast cancer. In certain embodiments the target molecule is a HER2 protein, or domain or region thereof.

According to frequently included embodiments, a method to evaluate target tissue by magnetic resonance imaging is provided as above, wherein identification of heterogenous hypointensity in the target tissue or lymph node is further indicative of malignancy of the subject tissue.

Frequently according to embodiments, a method to evaluate target tissue by magnetic resonance imaging is provided as above, further comprising assessing an image of the at least one lymph node for heterogenous architecture.

These and other embodiments, features, and advantages will become apparent to those skilled in the art when taken with reference to the following more detailed description of various exemplary embodiments of the present disclosure in conjunction with the accompanying drawings.

### BRIEF DISCUSSION OF THE DRAWINGS

FIG. 1 illustrates a biofunctional magnetic nanoparticle used as drug substance in the biofunctional magnetic nanoparticle solution according to embodiments.

FIG. 2 shows pre-contrast and post-contrast MR images of normal lymph nodes. The lymph nodes appear to be uniformly dark in post-contrast MR images, suggesting that it is normal.

FIG. 3 shows pre-contrast and post-contrast MR images of pathologically enlarged lymph nodes. The lymph nodes show heterogenous hypo-intensity in post-contrast MR images, suggesting that it is diseased. Some of the cortex is slightly darker than the pre-contrast image, which is uniform hypointensity, indicating that this region is not pathologically invaded.

FIG. 4 depicts illustrations of a lymph node with certain features. The left is a lymph node pre-contrast. The right is the observed pattern of homogeneous hypo-intensity after administration of the imaging agent on the same lymph node.

FIG. 5 depicts illustrations of a different lymph node with certain features. The left is the lymph node pre-contrast. The right is the observed pattern of heterogeneous hypo-intensity (“speckled”) after administration of the imaging agent on the same lymph node.

FIG. 6 depicts illustrations of lymph nodes with the observed pattern of heterogenous architecture with partial irregular darkening after administration of the imaging agent.

FIG. 7 depicts illustrations of lymph nodes with the observed pattern of both heterogenous architecture with partial irregular darkening and heterogeneous hypo-intensity (speckled”) after administration of the imaging agent.

FIG. 8 shows pre contrast and post contrast MR images of a lymph node showing heterogenous hypointensity on the post contrast T2 weighted image.

FIG. 9 shows pre contrast and post contrast MR images of a lymph node showing heterogenous architecture (with irregular darkening) on the post contrast T2 weighted image.

FIG. 10 shows pre contrast and post contrast MR images of a lymph node showing both heterogeneous hypo-intensity and heterogenous architecture on the post contrast T2 weighted image.

FIG. 11 shows pre contrast and post contrast MR images of a lymph node showing no contrast uptake, with an adjacent node showing homogenous hypointensity.

FIG. 12 depicts Magnetic Relaxometry and ICP-MS Analysis of BT474 and MCF7 cells exposed to imaging agent including anti-HER2 targeting ligand.

FIG. 13 depicts a Superparamagnetic Relaxometry Signal from High, Low and Negative HER2-Expressing Cells exposed to exemplary imaging agents specific for HER2.

FIG. 14 depicts Superparamagnetic Relaxometry Signal from High, Medium and Low HER2-Expressing Cells Exposed to the imaging agent including anti-HER2 targeting ligand.

FIG. 15 depicts Superparamagnetic Relaxometry Signal from High and Non-HER2-Expressing Cell Titrations Exposed to imaging agent including anti-HER2 targeting ligand.

FIG. 16 depicts Superparamagnetic Relaxometry Signal from BT474 cells, Lymphocytes and Peripheral Blood Mononuclear Cells Exposed to imaging agent including anti-HER2 targeting ligand.

FIG. 17 depicts Superparamagnetic Relaxometry Signal from HER2-positive Cell Implants Exposed to imaging agent including anti-HER2 targeting ligand, Pegylated Nanoparticle Control, and Free Antibody Competition.

FIG. 18 depicts Superparamagnetic Relaxometry Signal and Staining of BT474 and MCF7 Cell Tumors Exposed to imaging agent including anti-HER2 targeting ligand by Different Administration Routes.

FIG. 19 depicts Time Course Drainage of the imaging agent in Lymph Nodes Measured by MRX.

FIG. 20 depicts Presence of imaging agent after 24 hours in Resected Target Ancillary Lymph Nodes Demonstrated by Node Color (Top Image: dark brown/black color) and Prussian Blue Iron Staining.

FIG. 21 depicts Lymph Node Section Demonstrating the Presence of imaging agent by Prussian Blue Iron Staining in the Tumor Cells and Lymph Node Mouse Tissue.

### ABBREVIATIONS

CDRs: Complimentary Determining Regions
HER-2: Human Epidermal Growth Factor Receptor 2
MR: Magnetic Resonance
MRI: Magnetic Resonance Imaging
MRX: Magnetic Relaxometry

### DETAILED DESCRIPTION OF THE INVENTION

As used herein, “a” or “an” means “at least one” or “one or more.”

As used herein, the term “and/or” may mean “and,” it may mean “or,” it may mean “exclusive-or,” it may mean “one,” it may mean “some, but not all,” it may mean “neither,” and/or it may mean “both.”

As used herein, the term “antibody” (Ab) refers to an immunoglobulin molecule that specifically binds to, or is immunologically reactive with, a particular antigen—here, HER2. Antibodies comprise complementarity determining regions (CDRs), also known as hypervariable regions, in both the light chain and heavy chain variable domains. The more highly conserved portions of the variable domains are called the framework (FR). As is known in the art, the amino acid position/boundary delineating a hypervariable region of an antibody can vary, depending on the context and the various definitions known in the art. Some positions within a variable domain may be viewed as hybrid hypervariable positions in that these positions can be deemed to be within a hypervariable region under one set of criteria, while being deemed to be outside a hypervariable region under a different set of criteria. One or more of these positions can also be found in extended hypervariable regions. The variable domains of native heavy and light chains each comprise four FR regions, largely by adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies. See Kabat et al., Sequences of Proteins of Immunological Interest (National Institute of Health, Bethesda, Md. 1987). As used herein, numbering of immunoglobulin amino acid residues is done according to the immunoglobulin amino acid residue numbering system of Kabat et al. unless otherwise indicated.

Antibodies and/or binding fragments composing the anti-HER2 antibodies generally comprise a heavy chain comprising a variable region (V_{H}) having three complementarity determining regions (“CDRs”) referred to herein (in N→C order) as V_{H} CDR#1, V_{H} CDR#2, and V_{H} CDR#3, and a light chain comprising a variable region (V_{L}) having three complementarity determining regions referred to herein (in N→C order) as V_{L} CDR#1, V_{L} CDR#2, and V_{L} CDR#3. The amino acid sequences of exemplary CDRs, as well as the amino acid sequence of the V_{H} and V_{L} regions of the heavy and light chains of exemplary anti-HER2 antibodies and/or binding fragments that can be included in antigen binding moieties are provided herein. Specific embodiments of anti-HER2 antibodies include, but are not limited to, those that comprise antibodies and/or binding fragments that include these exemplary CDRs and/or V_{H} and/or V _{L} sequences, as well as antibodies and/or binding fragments that compete for binding HER2 with such antibodies and/or binding fragments.

Antibodies may be in the form of full-length antibodies, bispecific antibodies, dual variable domain antibodies, multiple chain or single chain antibodies, surrobodies (including surrogate light chain construct), single domain antibodies, camelized antibodies, scFv-Fc antibodies, and the like. They may be of, or derived from, any isotype, including, for example, IgA (e.g., IgA₁ or IgA₂), IgD, IgE, IgG (e.g., IgG₁, IgG₂, IgG₃ or IgG₄), IgM, or IgY. In some embodiments, the anti-HER2 antibody is an IgG (e.g., IgG₁, IgG₂, IgG₃ or IgG₄). Antibodies may be of human or non-human origin. Examples of non-human origin include, but are not limited to, mammalian origin (e.g., simians, rodents, goats, and rabbits) or avian origin (e.g., chickens).

A Fab fragment contains the constant domain of the light chain and the first constant domain (CH₂) of the heavy chain. Fab′ fragments differ from Fab fragments by the addition of a few residues at the carboxyl terminus of the heavy chain CH₂ domain including one or more cysteines from the antibody hinge region. F(ab′) fragments are produced by cleavage of the disulfide bond at the hinge cysteines of the F(ab′)₂ pepsin digestion product. Additional chemical couplings of antibody fragments are known to those of ordinary skill in the art. Fab and F(ab′)₂ fragments lack the Fc fragment of intact antibody, clear more rapidly from the circulation of animals, and may have less non-specific tissue binding than an intact antibody.

An “Fv” fragment is the minimum fragment of an antibody that contains a complete target recognition and binding site. This region consists of a dimer of one heavy and one light chain variable domain in a tight, non-covalent association (V_{H}-V_{L} dimer). It is in this configuration that the three CDRs of each variable domain interact to define an antigen binding site on the surface of the V_{H}-V_{L} dimer. Often, the six CDRs confer antigen binding specificity upon the antibody. However, in some instances even a single variable domain (or half of an Fv comprising only three CDRs specific for a target) may have the ability to recognize and bind antigen, although at a lower affinity than the entire binding site.

“Single-chain Fv” or “scFv” antibody binding fragments comprise the V_{H} and V _{L} domains of an antibody, where these domains are present in a single polypeptide chain. Generally, the Fv polypeptide further comprises a polypeptide linker between the V_{H} and V_{L} domains which enables the scFv to form the desired structure for antigen binding.

As used herein, “targeting ligand” refers to an antibody in the broadest sense that term is used herein and in the art, or another moiety adapted to specifically bind a predetermined target molecule.

As used herein, “MR imaging” refers to magnetic resonance imaging or MRI. The terms MRI and “MR imaging” are used interchangeably herein.

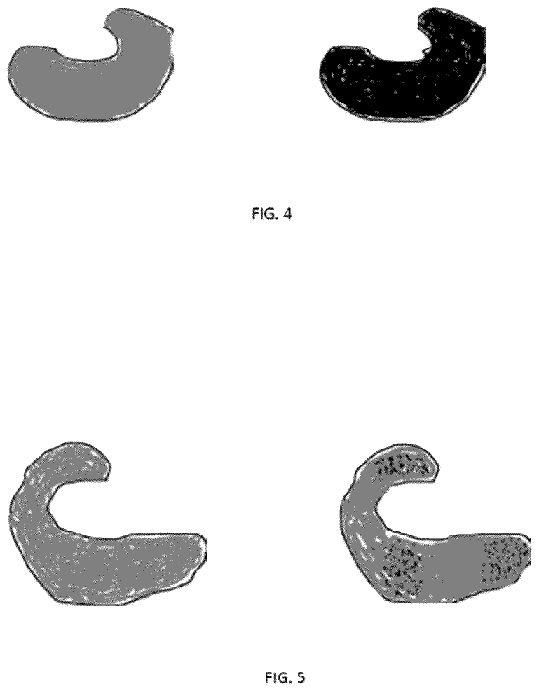

As used herein, “heterogenous hypo-intensity” refers to a spotted molecular signature in MRI images in a heterogenous hypo pattern, which has been found by the inventors as indicative of the sufficient imaging agent specifically bound to a target molecule to be detected by MRI, e.g., in vivo. FIG. 4 depicts an illustration of what is intended by the term heterogenous hypo-intensity when viewed in an exemplary target tissue. Other types of target tissues in a subject including and additional to lymph nodes are specifically contemplated for imaging according to the methods described herein.

As used herein “heterogenous architecture” refers to a signature in MRI images with heterogenous appearances of light and dark, which has been hypothesized by the inventors as indicative of sufficient imaging agents bound to target molecules to be detected by MRI, e.g., in vivo. FIG. 6 depicts an illustration of what is intended by the term “heterogenous architecture” when viewed in an exemplary target tissue.

As used herein “imaging agent” refers to a biofunctional magnetic nanoparticle solution formulated for safe in vivo use in a human comprised of an iron oxide nanoparticle, often between 10 and 150 nanometers in diameter, functionalized with a targeting ligand, and with a poly dispersive index (PDI)<0.2. The imaging agent is often referred to as a complete solution or the functionalized nanoparticle contained therein. The prepared and functionalized nanoparticle, including the magnetic nanoparticle, organic acid, polymer and targeting ligand as described herein is also referred to as a biofunctional magnetic nanoparticle.

Cancer diagnosis and staging requires a combination of clinical classification and invasive pathologic assessment. For breast cancer patients, staging of the cancer requires determining if the primary tumor cells have spread to lymph nodes. Based on palpation or imaging techniques such as ultrasound, PET, CT, or MRI, nodes may be clinically suspicious for metastasis and undergo biopsy for pathological confirmation.

For many breast cancer patients, current imaging methods cannot reliably detect micro and macro metastases and the lymph nodal assessment involves surgical resection of 2 to 4 of the sentinel lymph nodes (SLN) and, depending on the degree of tumor infiltration determined by laboratory pathology analyses, complete dissection of the axillary lymph nodes (ALN). Despite only approximately 25% of clinically node negative breast cancer patients having lymph nodal involvement, the standard of care today still results in all patients with node negative diagnosis by standard of care imaging undergoing a surgical procedure to confirm if there has been metastatic spread. This means that the majority of patients undergo a surgical procedure unnecessarily, resulting in avoidable morbidities such as lymphedema, intercostobrachial neuralgia, regional cellulitis or infection, as well as limiting treatment options should cancer recurrence occur. A non-invasive method to confirm lymph nodal involvement has been found to significantly improve patient care by avoiding the cost and risk of surgical biopsy procedures for the majority of patients and eliminate the significant concomitant morbidities associated with nodal resection.

While not intending to be bound by any theory of operation, it has been found that:
The presently described imaging agents are specific for HER2-expressing cancer cells;
The imaging agent's utility in providing a tumor-specific contrast agent in MRI and to improve its detection sensitivity and specificity;
The imaging agents do not have significant non-specific binding with non-tumor cells such as dissociated human cadaver lymph node cells and peripheral blood mononuclear cells (PBMCs);
Uptake of the imaging agent in the lymph nodes when administered by intraperitoneal and peri-tumor injections in mouse models; and
A time period, e.g., up to or over 16 hours is preferred from the time of imaging agent administration to surgery measurement in order to obtain optimal signal from the imaging agent.

According to the presently described protocols, a single dose of 30 mg (or 20 mg) imaging agent (based on iron oxide weight) is assessed. Based on the assumption that minimally 1% of material (200 to 300 μg imaging agent) is able to distribute to the lymph nodes via injection peritumorally in the breast. Overall, the selected dose translates (per FDA Guidance “Estimating the Maximum Safe Starting Dose in Initial Clinical Trials for Therapeutics in Adult Healthy Volunteers” 6) to a significantly reduced dose of iron oxide (0.3 to 0.5 mg/kg) per body weight compared to nonclinical studies.

In the FIH study, a single dose of 30 mg (or 20 mg) imaging agent (based on iron oxide weight) was assessed. Overall, the selected dose translates (per FDA Guidance “Estimating the Maximum Safe Starting Dose in Initial Clinical Trials for Therapeutics in Adult Healthy Volunteers” 6) to a significantly reduced dose of iron oxide (0.3 to 0.5 mg/kg) per body weight compared to nonclinical studies.

This disclosure relates to biofunctional magnetic nanoparticle solutions and their use as test reagents in magnetic resonance imaging. A drug substance is provided herein formulated in a solution for injection (e.g., via parenteral administration, including intravenous, subcutaneous, or intramuscular, for example, subareolar, peritumoral, etc.) in a subject comprising biofunctional magnetic nanoparticles.

According to the methods of the present disclosure, human subjects with suspected or confirmed breast cancer are assessed for nodal disease or nodal metastases, including axillary nodal disease. According to such methods the human subjects receive an injection of the imaging agent according to embodiments described herein in, for example, via parenteral administration, including intravenous, subcutaneous, or intramuscularly, for example, in the subareolar or peritumor region. Thereafter, the human subjects receive MR imaging of a target region, for example the axilla region. MR imaging can be T1-weighted imaging sequence or T2/T2* weighted imaging sequence, and the resulting images are evaluated by radiologists. The present methods provide for imaging-based diagnoses without the need for biopsies, thereby reducing the need for additional hospital visits and surgeries, which is the current standard of care.

The imaging agent according to embodiments herein is a biofunctional magnetic nanoparticle solution, with the nanoparticle having an iron core coated in organic coating, such as oleic acid, which is then coated with a polymer, such as poly(maleic anhydride-alt-octadecene), then conjugated with polyethylene glycol polymers and an antibody specific to the type of cancer present in the human subject, such anti-HER-2 antibody, trastuzumab, in subjects with HER-2 breast cancer. Other organic substances may be used for coating, such as dextran. Other polymers for coating and/or conjugation may be used. Other antibodies specific to different tumors are contemplated. The imaging agent is provided as a solution for parenteral administration, such as via injection. In MR imaging, targeting ligand bearing nanoparticles in the imaging agent bind with a tissue of interest and thereby provide for an image that is discernable from tissues not of interest based on certain features of the image. These features include an area of heterogenous hypo-intensity in the tissue of interest in the MRI images, as evaluated by a radiologist. The exemplary targeting ligand used in the study described herein is an anti-HER-2 antibody that is specific for the HER-2 protein that has a known association with the presence of breast cancer. In axilla lymph nodes where cancer has metastasized, the use of an imaging agent capable of providing discernable imaging differences is of high value, since lymph nodes are small and relying on morphology assessment alone has low accuracy.

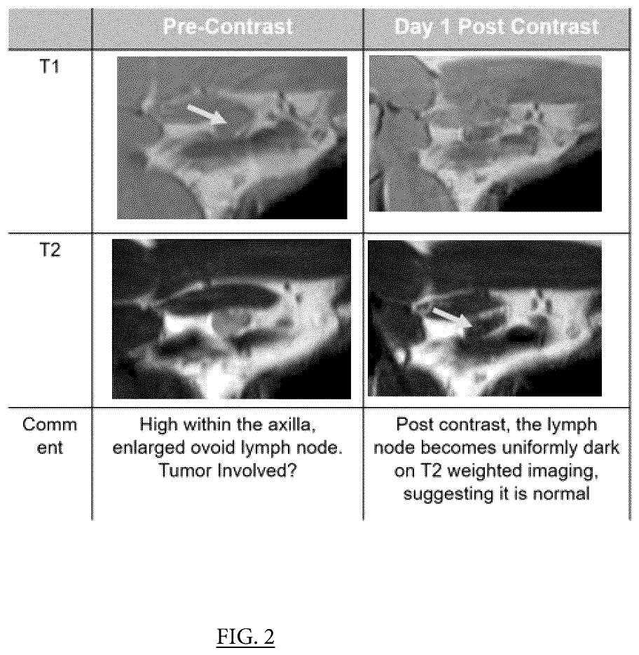

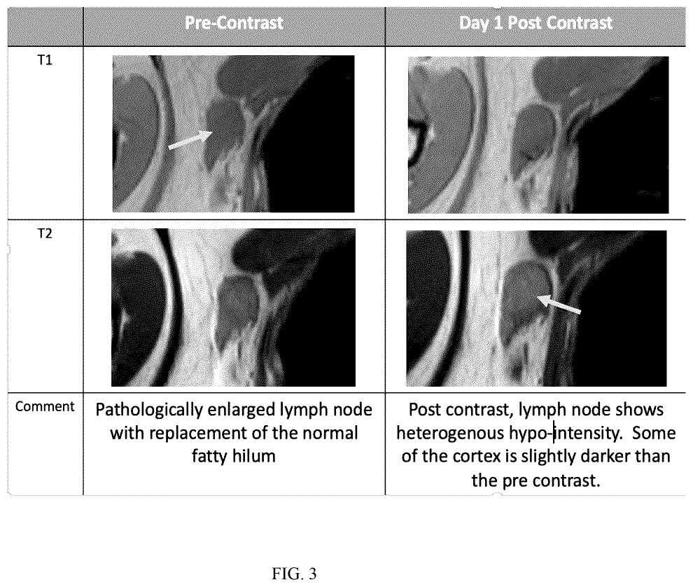

According to certain embodiments described herein, after receiving an injection of the imaging agent as provided herein, a human subject receives MR imaging to evaluate axilla lymph nodes, where the imaging agent would drain to, among other regions. In normal lymph nodes, the images of the lymph nodes appear uniformly dark, as depicted in FIG. 2. In diseased lymph nodes of HER-2 breast cancer patients, the images of the lymph nodes show heterogenous hypo-intensity, as depicted in FIG. 3.

By reading the MR images of axilla lymph nodes injected with the imaging agent according to embodiments, radiologists can determine if a lymph node has HER-2 breast cancer tissues present based on identification of areas of heterogenous hypo-intensity in the MRI images of the tissue of interest.

According to an exemplary embodiment, imaging schedule Baseline MRI assessments are performed prior to imaging agent administration and one or two additional timepoint post imaging agent administration, as follows: Visit 2, Day 1—PRIOR to imaging agent injection; Visit 3, Day 2 within 18-30 hour and Visit 5, Day 4 within 66-78 hour time-window or Visit 3, Day 2/3, within the 18-78 hour time-window after imaging agent injection. Imaging agent administration is performed by a surgeon or a radiologist following completion of the baseline MRI scan on Day 1. Administration of imaging agent can be performed in the radiology department or other suitable setting. As a peritumoral injection may involve additional interventional ultrasound (US) guidance appropriate ultrasound equipment should be available to support administration.

In this schedule it has been found useful to match the node or nodes removed or biopsied with the node or nodes imaged postdose in order to compare postdose MRI scans and pathology results of the removed or biopsied nodes in terms of tumor presence. To that end, the suspicious node needs to be localized by a clip that is magnetic resonance-compatible for same-day insertion as the MRI, using the imaging modality (e.g., ultrasound) available at the site. Clip insertion can be performed any time after informed consent and before postdose MRI.

During imaging, the subject is often positioned supine on the spine coil, off centered so that the side (location) of breast cancer pathology is centered within the magnet. For example, if the right breast (affected side) has the suspicious lesion then the subject should be offset to the left side of the spine coil so that the right axilla is centered on the spine coil, or as close as practicable, depending on subject's size. The highest density body array coil available is placed over the affected side axilla region of the subject. The subject's arm on the affected side is preferably placed firmly by their side eliminating any spaces between the arm and the body. The other arm can be placed either by their side also, or above the head as is preferred. Additional immobilization sponges can be used to assist with limiting subject motion.

The following combination of image sequences are often utilized at each MRI acquisition (estimated total MRI acquisition time is approximately 20 minutes):
Localizers
Localization of the lymph nodes 1 using:
   T1 gradient echo
   T2 turbo spin echo (TSE)
   T1 multi station gradient echo (VIBE) for nanoparticle detection
   T2* gradient recalled echo (GRE) for nanoparticle detection, whenever feasible

Additional sequences may be performed to meet the site's standard of practice and optional imaging parameters can be applied at the discretion of the site as long as documented.

Field strength preferably is often 1.5 T. Only if needed, as a second option, a field strength of 3 T could be used with an adjusted acquisition protocol.

### Contrast Material

An exemplary imaging agent used for the MR imaging according to the present invention is a biofunctional magnetic nanoparticle solution, which is also referred to herein using the imaging agent. Exemplary biofunctional magnetic nanoparticles in the imaging agent are composed of an iron oxide nanoparticle coated with a surfactant (e.g., oleic acid) and a layer composed of a carboxyl group functionalized with polymer poly(maleic anhydride-alt-octadecene) (POMA). This coated nanoparticle is conjugated with polyethylene glycol polymers and anti-HER-2 antibody, trastuzumab. An exemplary imaging agent is generally comprised of an optionally filtered aqueous nanoparticle solution in 0.9% NaCl containing 0.05% polysorbate-20.

Overall, the exemplary imaging agent comprises an injectable formulation comprised of SPIONs conjugated to an anti-HER2 antibody. The NPs consist of the iron oxide spherical core (24 to 28 nm in diameter) which is coated with an amphiphilic polymer (poly maleic anhydride-alt-1-octadecene [POMA]), which serves as a linker for the covalent conjugation of trastuzumab (anti-HER2 antibody) and polyethylene glycol (PEG). The core iron oxide NPs and POMAc-functionalized NPs were manufactured by nanoComposix (San Diego, CA).

The final antibody conjugated NP drug substance was produced as a colloidal solution containing the NPs (˜10 mg/mL based on iron oxide) in sterile (0.9%) saline and 0.05% polysorbate 20 solution for stability. The final imaging agent formulation is identical to the bulk drug substance solution. The imaging agent bulk material was transferred to PCI Pharma Services (Melbourne, Australia) and sterile filtered (0.22 μm filter) into 2 mL sterile vials (Crystal Zenith vial, West Pharma) containing 1.3 mL of imaging agent solution and capped with sterile 13 mm stoppers and aluminum caps following GMP manufacturing guidelines. Sterility analysis was performed by a GMP analytical lab (Eurofins) in Australia.

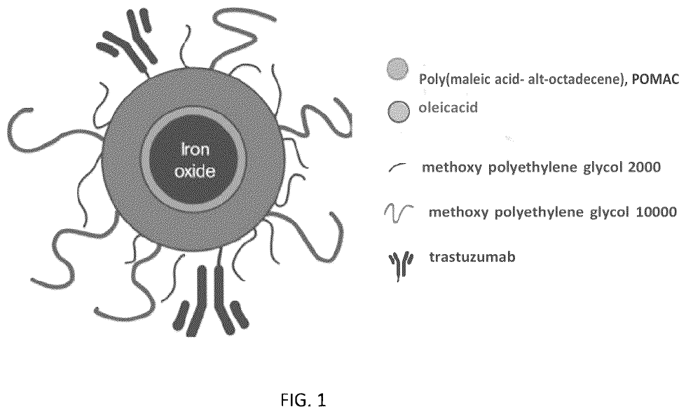

The primary structure of the POMAc-NP conjugated with polyethylene glycol polymers and trastuzumab is illustrated in FIG. 1. This primary structure comprises an iron oxide core coated with a layer of oleic acid, which is then coated with a layer of poly(maleic acid-alt-octadecene), with methoxy polyethylene glycol 2000, methoxy polyethylene glycol 10000 and trastuzumab conjugated to the layer of poly(maleic acid-alt-octadecene).

The biofunctional magnetic nanoparticle solution used in this invention is an aqueous, dark brown to black, colloidal solution of biofunctional magnetic nanoparticles as illustrated in FIG. 1 and supplied as drug product for parenteral administration, including intravenous, subcutaneous, or intramuscularly, for example, subareolar or peritumoral injection. It is supplied as a 1 mL fill, single-use, colloidal solution formulation at 7.5 mg/mL Fe equivalent strength, in aseptically filled and finished vials. The imaging agent in the drug product is formulated as an aqueous nanoparticle solution in 0.9% NaCl containing 0.05% polysorbate 20. The product is stored at 2° C. to 8° C.

The maximum dose volume delivered to humans to date is 3 mL at 7.5 mg/mL Fe equivalent, i.e., a 22.5 mg Fe equivalent dose of the imaging agent. This dose volume and dose strength is also referred as 3 mL of the calculated dose of 10 mg/mL Fe₃O₄ equivalent, or a maximum dose of 30 mg of Fe₃O₄ (calculated) or 30 mg of the imaging agent.

The present imaging agent as an MRI contrast agent and with its tumor targeting properties has the potential to enhance the diagnostic accuracy of clinical nodal assessment when used alone or as an add-on to current imaging methods in HER-2-positive breast cancer patients, providing the rationale for evaluating the present imaging agent in this specific patient population.

### Exemplary Studies

### Cell Binding and Sensitivity of the Imaging Agent HER2 Test Reagent In Vitro

Study Objective: A cell binding study was performed to verify the sensitivity of imaging agent for targeting HER2-positive tumor cells.

Method: The study was conducted using the BT474 breast cancer cell line known to express high levels of HER2, and MCF7 cells known to be a HER2-low or negative cell line. Cells were seeded in 6-well cell culture plates 24 hours prior to the addition of the 100 μg (based on iron oxide) of imaging agent. After a 24-hour incubation period, the cells were washed to remove unbound imaging agent and harvested for an SPMR measurement.

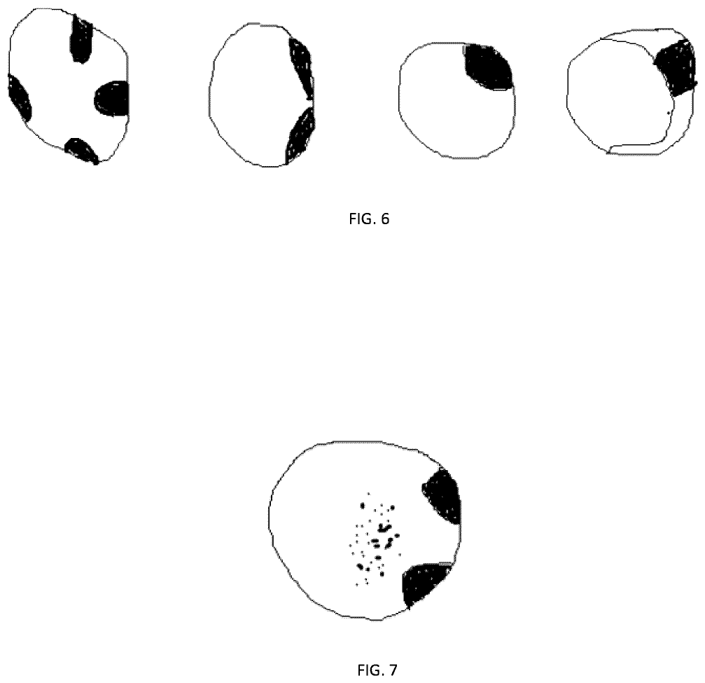

Results: Titration experiments (varying the number of cells from 0.25×106 to 2×106) indicated that the test reagent was detectable in BT474 cells at the lowest titration of 0.25×106 (FIG. 5, left panel). Results were confirmed by inductively coupled plasma mass spectrometry (ICP-MS) analysis to measure the amount of iron present in each titration (FIG. 5, right panel).

Conclusion: The imaging agent can bind specifically to HER2-positive cells (BT474). These signals are further verified using ICP-MS by measuring amount of iron presence.

### Specificity and Selectivity of the Imaging Agent Containing an Anti-HER2 Targeting Ligand In Vitro

Study Objective: An in vitro cell binding study was undertaken to assess the specificity of the imaging agent for targeting high, low and negative HER2-expressing tumor cells.

Method: Binding to 1 high expressing HER2 cell line (BT474) was compared to 2 cell lines that are low or non-expressing (MCF10, MCF7, OVCAR), and 1 cell line that was HER2-positive but non-responsive to Herceptin (JIMT). In this study, 100 μg of imaging agent was incubated for 24 hours with 1×106 cells of each type and the binding was assessed by detection of SPMR signal.

Results: imaging agent was able to generate an appreciably measurable signal by SPMR with the high HER2 expressing cancer cells (BT474) but did not produce measurable signals with low or negative HER2 expressing cells (MCF10, JIMT, OVCAR and MCF7) (FIG. 6).

Conclusion: This study demonstrated that the imaging agent has high specificity and selectivity for targeting HER2-positive tumor cells.

### Cell Selectivity and Specificity Studies

Study Objective: A cell binding study was conducted to assess the specificity of imaging agent for targeting high, medium and low or negative HER2-expressing tumor cells, with and without exposure to free anti-HER2 antibody (compete).

Method: In this study, 100 μg of imaging agent was incubated for 24 hours with 1×106 cells from multiple high expressing HER2 positive (IHC 3+) cell lines, but with varying degrees of HER2 expression (SKBR3, BT474, HCC1954), as well as a medium expressing (IHC 2+) cell line (ZR75), and 1 of low or non-expression (MCF7). The studies included a competition study group in which all of the cells were exposed to free anti-HER2 antibody (100× excess compared to the Herceptin on the NPs) prior to incubation with the imaging agent. Binding was assessed by detection of SPMR.

Results: The results also demonstrated that the imaging agent and SPMR signal can be competed out by free antibody (FIG. 7).

Conclusion: The imaging agent is highly specific and selective to binding of cells with HER2 expression.

### Additional Cell Selectivity and Specificity Studies

Study Objective: An additional cell binding study was conducted to assess the effect of tumor cell titration on the level of imaging agent binding.

Method: High HER2-expressing cell lines (SKBR3, BT474 and HCC1954) and a non-HER2 expressing cell line (MDA-MB231) were titrated and incubated with 100 μg of imaging agent for 24 hours. The cell numbers assessed were 2.5×106, 5.0×106 and 10×106 cells. Binding was assessed by detection of SPMR signal and such signals were confirmed by the measurement of iron content using ICP-MS.

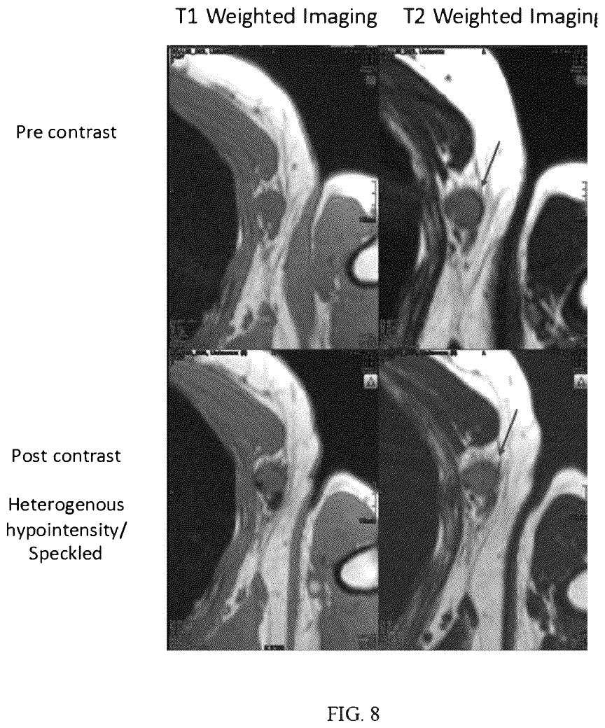

Results: Titrating high-and non-HER2 expressing cell lines with imaging agent demonstrated a distinct and direct correlation between cell number and SPMR signal (FIG. 8, left panel). These results were further confirmed by the corresponding iron content using ICP-MS (FIG. 8, right panel).

Conclusion: The SPMR signal is directly related to the number of labeled cells by imaging agent and this signal is directly related to amount of imaging agent presence in the sample.

### Non-Specific Interaction of Imaging Agent with Lymphocytes and Peripheral Blood Mononuclear Cells In Vitro

Study Objective: As the imaging agent's intended use is to detect HER2-positive tumor cells in lymph nodes, a study was conducted to determine whether there is non-specific binding of imaging agent with lymphocytes and PBMCs that would cause an interfering SPMR signal.

Method: Human cadaver lymph nodes were purchased from a commercial vendor and the lymph node cells were dissociated. Once isolated, 10×106 of the dissociated lymphocytes were incubated with 100 μg of imaging agent for 24 hours. imaging agent was also incubated with 1×106 of HER2-positive cells (BT474), and with a co-culture both cell types (1×106 BT474 and 10×106 lymphocytes). In addition, imaging agent was incubated with 10×106 PBMCs. Binding was assessed by detection of SPMR signal.

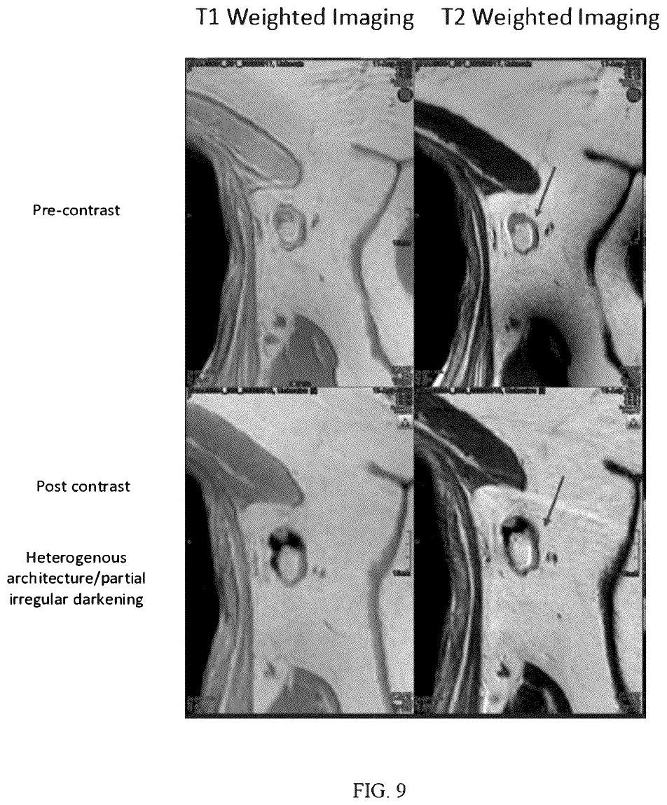

Results: An SPMR signal was generated from the imaging agent-incubated lymphocytes, indicating some degree of non-specific interaction with lymphocytes. An SPMR signal was also generated from the imaging agent-incubated HER2-expressing cells that was approximately 3-times higher compared to the non-specific signal from the lymphocyte culture (FIG. 9, left panel). The imaging agent-incubated PBMCs did not generate an SPMR signal, indicating there was no interaction of imaging agent with PBMCs (FIG. 9, right panel).

Conclusion: Taken together, these results demonstrate that the imaging agent is unlikely to produce a significant false positive signal due to non-specific interaction with non-tumor cells in lymph nodes and in blood.

### Tumor Detection and Specificity of the Imaging Agent in an In Vivo Xenograft Tumor Model in Mice

Study Objective: To determine if the imaging agent can target HER2+ tumor in vivo and generate sufficient signal for SPMR detection.

Method: Female athymic nude mice were implanted with 3×106 HER2-positive (BT474) cells subcutaneously in the flank region of the body. After 6 to 10 weeks, palpable tumors ranging from 0.125 cm3 to 1 cm3 in size had developed. Multiple different routes of administration including intraperitoneal, peritumoral, and intravenous injection into the tail vein were used to deliver 400 μg of imaging agent. pegylated NPs (same construction as imaging agent but without the anti-HER2 antibody) were employed as a control vehicle. An in vivo competition study was also conducted by pre-injecting 1 mg of free anti-HER2 antibody via the tail vein 24 hours prior to imaging agent delivery. Mice were euthanized 24 hours post-dose. Tumor and other major organs were resected and an ex vivo SPMR measurement was taken with the excised tissue, in addition to measurement of iron content using ICP-MS.

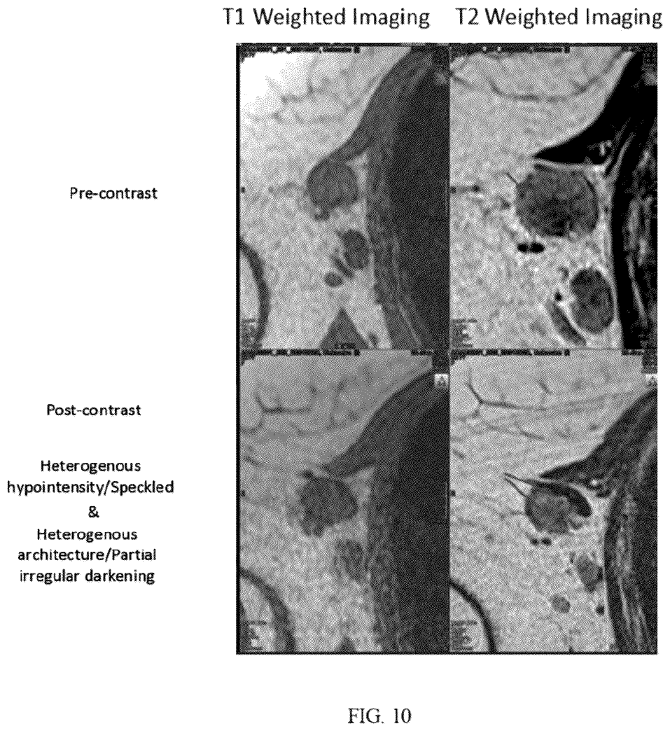

Results: The BT474 tumor cells exposed to imaging agent generated a greater SPMR signal compared to the tumor cells exposed to the control vehicle (FIG. 10). Additionally, the pre-injected free antibody outcompeted the imaging agent resulting in little to no SPMR signal.

Uptake of imaging agent in the xenograft tumor was further confirmed by measurement of iron content using ICP-MS. Quantitative analysis of the SPMR signal and ICP-MS suggested that, on average, approximately 2 to 5 μg of the imaging agent was present in the tumors, compared to levels that were below the ICP-MS detection limit for the control vehicle.

Conclusion: The imaging agent can bind to the tumor cells in vivo and generate specific signal measurable by MRX instrument.

### Additional Tumor Detection and Specificity of the Imaging Agent in in In Vivo Dual-Flank Xenograft Tumor Model in Mice

Study Objective: As a follow up study, a dual-flank tumor model was used to further demonstrate that imaging agent can bind to HER2 positive tumor cells but not to HER2 negative tumor in vivo and generate specific signal via various delivery routes.

Method: A dual-flank tumor model was generated by implanting 3×106 BT474 (HER2-positive) and 1×106 MCF7 (HER2-low or non-expressing, MCF7 is faster growing cell than BT474) cancer cells on each flank of female athymic nude mice. Multiple different routes of administration including intraperitoneal, peritumoral, and intravenous injection into the tail vein were used to deliver 400 μg of imaging agent. Pegylated NPs (same construction as imaging agent but without the anti-HER2 antibody) were employed as a control vehicle. Mice were euthanized 24 hours post-dose. Tumor and other major organs were resected and an ex vivo SPMR measurement was taken with the excised tissue, in addition to Prussian blue and anti-Herceptin staining to localize imaging agent within the tumors.

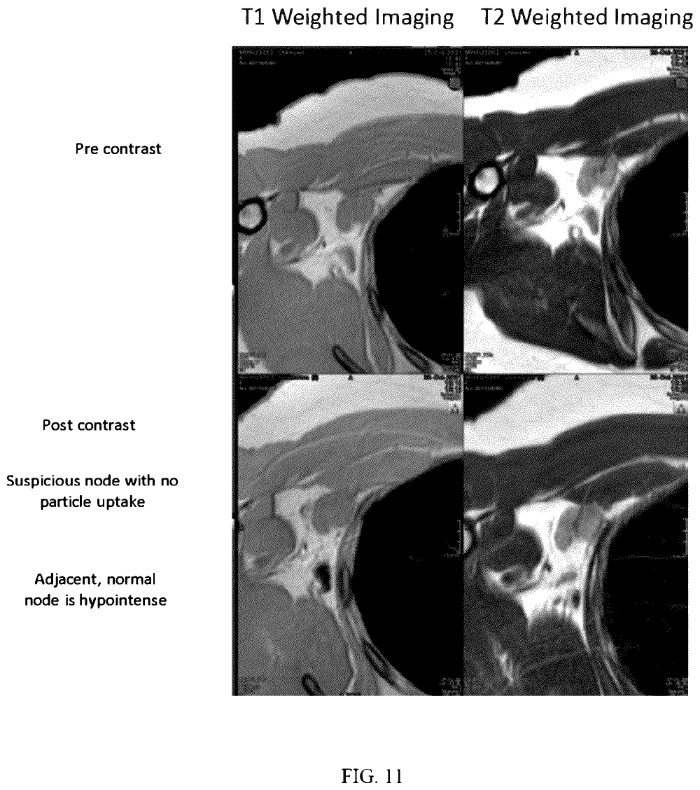

Results: All administration routes trialed resulted in a significantly higher SPMR signal in the BT474 tumors compared to the MCF7 tumors (FIG. 11, left panel). Presence of imaging agent was located mostly on the outer portion of the tumor, as indicated by Prussian blue staining (blue staining) and anti-Herceptin staining (brown staining) (FIG. 11, right panel).

Conclusion: These results demonstrate that the specificity of imaging agent to target HER2-positive tumor cells is maintained in vivo and is detectable by SPMR measurement. Imaging agent clearance is predominately through the liver and spleen, as would be expected from the scientific literature for a NP in the similar size range.

### Distribution of the Imaging Agent to Lymph Nodes in Mice

Study Objectives: A mouse-based study was designed and conducted to assess the time required to drain imaging agent from the lymph nodes when injected into the distal mammary pad.

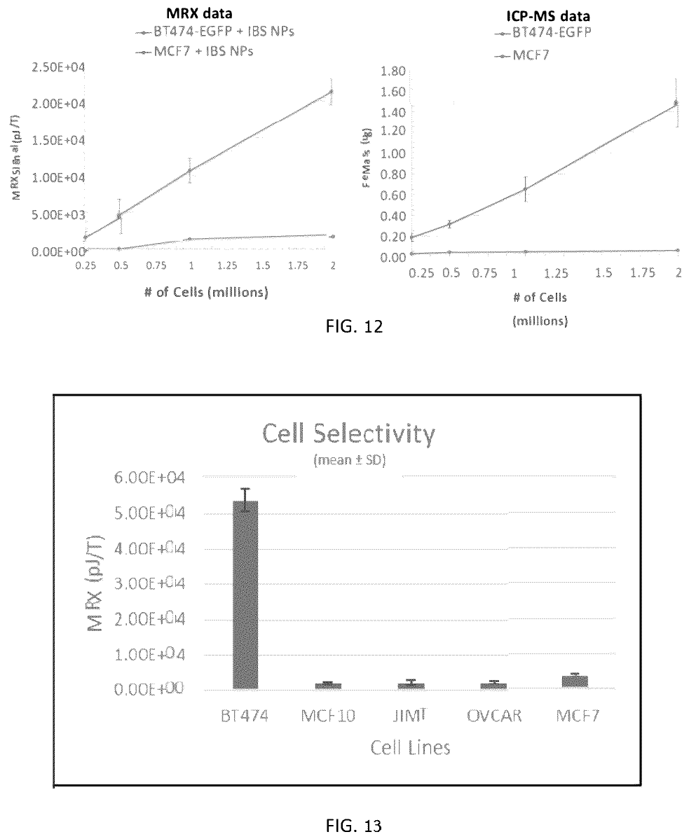

Method: 433 μg of imaging agent was injected into right nipple/areola of the fourth abdominal mammary pad of 9 female athymic naïve nude mice. The axillary and inguinal lymph nodes were resected at 24, 48 and 72 hours after the injection (3 mice for each time point). Visual inspection of the color of the resected nodes was performed 24 hours after injection to determine the presence of imaging agent within the node and Prussian blue staining was performed to localize imaging agent within the node. Resected lymph nodes were measured ex vivo by the MRX instrument. Measurement of iron content within each node was also performed using ICP-MS to confirm the MRX results (FIG. 12).

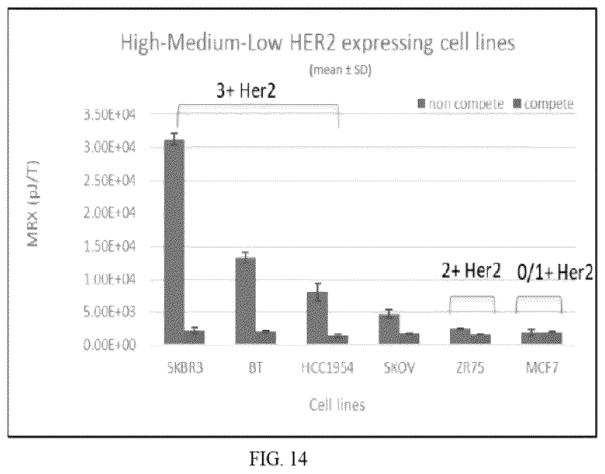

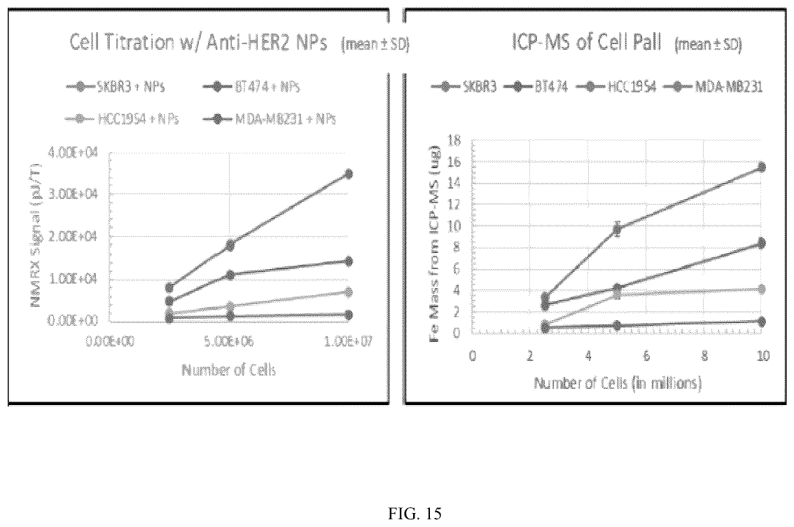

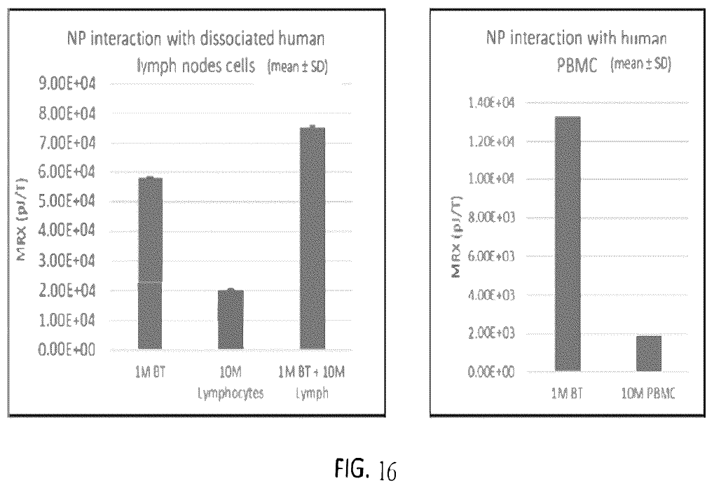

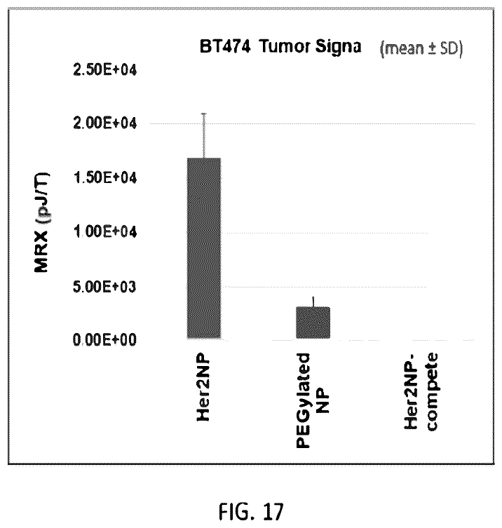

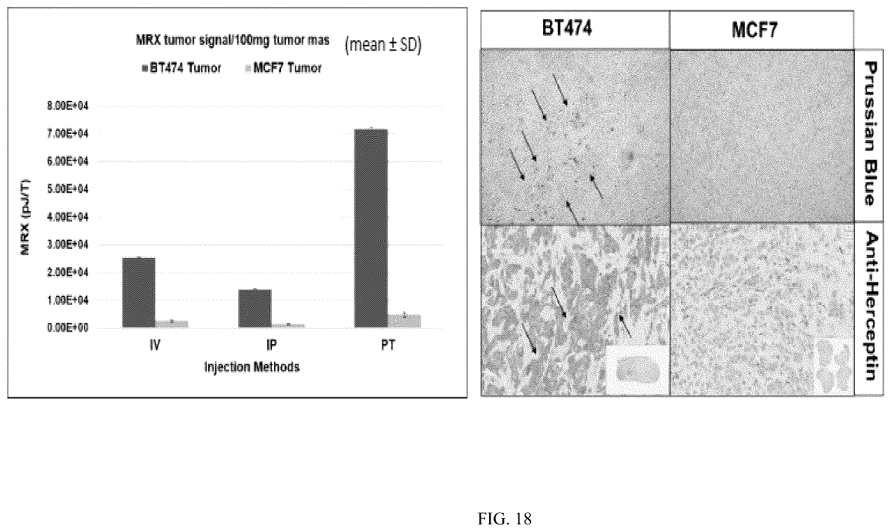

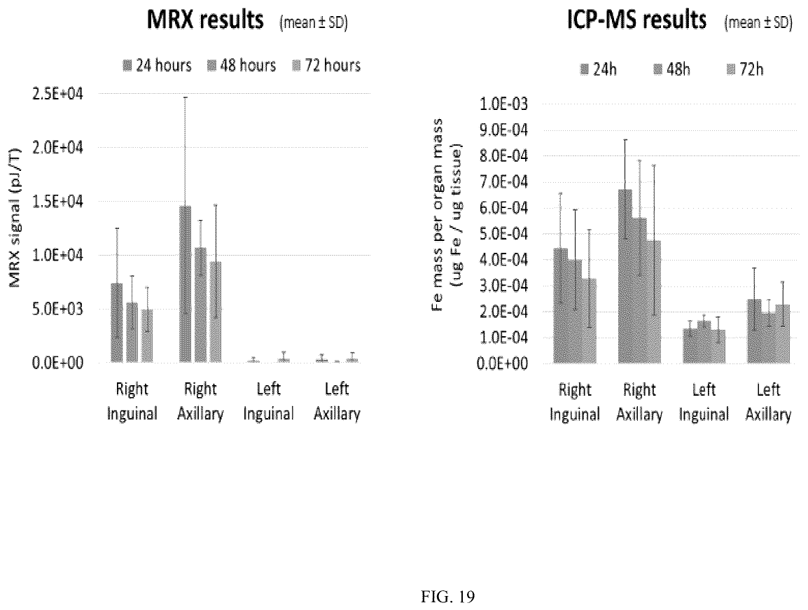

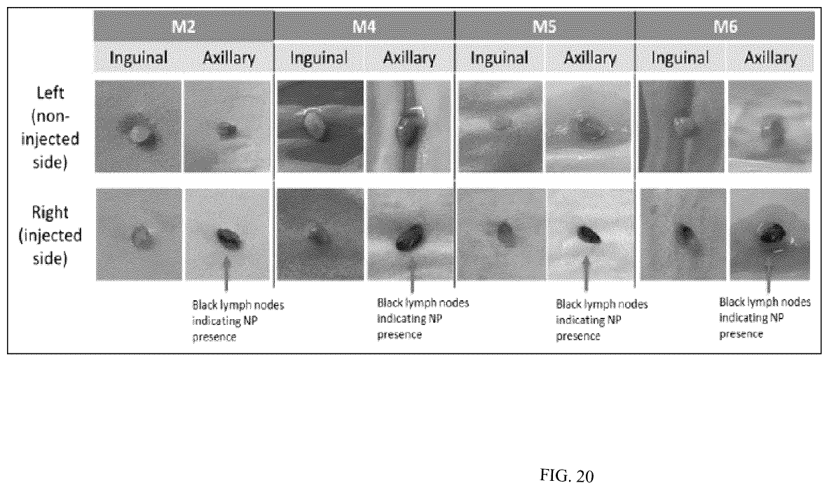

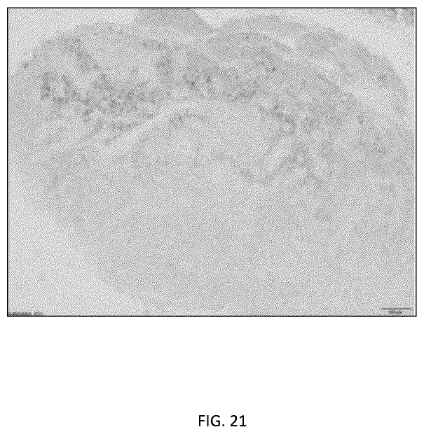

Results: The target ALNs appeared distinctively darker, with brown/black coloring, compared to the inguinal lymph nodes from the same side and the ALNs from the opposite side of the body (FIG. 13). The presence of imaging agent in the ALNs was confirmed by Prussian blue staining which showed that imaging agent was predominately located in the sinus of the lymph tissue, indicating proper drainage (FIG. 14).

ICP-MS further confirmed the presence of imaging agent in the ALNs by detecting elemental iron). On average, 6 μg of Fe was detected by ICP-MS for the ALN and 1 μg for inguinal lymph nodes from the same injection of the mice.

After 72 hours, majority of imaging agent drained away from the lymph nodes, particularly in the inguinal lymph nodes which were the closest to the injection sites (FIG. 12).

Conclusions: The imaging agent are drained more efficiently from the lymph nodes at 72 hours, therefore minimizing potential non-specific signal of MRI/MRX readout.

### MRI Imaging Using the Imaging Agent—First Cohort

The imaging agent was utilized in in vivo MRI studies in six (6) patients as follows.

Human participant selection was based on the following criteria: participants were diagnosed with HER-2-positive primary breast cancer, who were scheduled to undergo preoperative clinical assessment of the axillary nodal disease followed by core biopsy or pathological confirmation. Participants with node-suspicious by routine axillary assessments took part in this study. Exclusion criteria were known inflammatory breast cancer and prior surgical axillary procedure including sentinel lymph node biopsy (SLNB) or axillary lymph node dissection (ALND) or previous radiation on the ipsilateral side of the breast cancer primary. Participants, who in the judgment of the Investigator, have a likelihood of lymph node metastasis and are either scheduled for sentinel lymph node biopsy (SLNB) and/or axillary lymph node dissection (ALND) (Arm 1) or were scheduled for biopsy of suspicious lymph nodes (Arm 2), or have already had a biopsy of suspicious lymph nodes (Arm 3), were selected for enrolment.

All eligible participants received a single dose of 22.5 mg Fe equivalent of drug substance into the subareolar interstitial tissue or area near and around the primary tumor on Study Day 1. Baseline MRI assessments were performed within 3 days before imaging agent administration (the imaging agent being the biofunctional magnetic nanoparticle solution described herein), and a second MRI was performed within 18-30 hours and a third MRI was performed within 66-78 hours, post-imaging agent administration and before any neoadjuvant therapy. Either whole lymph node(s) or lymph node tissue from a core biopsy was obtained for histopathology assessment. These specimens are also used for ex vivo MRX measurements.

The study consisted of a screening, baseline, imaging, and follow-up period. Participants undertook a Screening Visit between Day −28 and Day −1 to determine eligibility in the study. Each participant was dosed in the baseline period after obtaining baseline MRI scan or on Day 1 in the imaging period. Postdose MRI scans and pathology specimens were obtained in the imaging period. Data acquisition and assessments include safety assessments and safety labs; image acquisition, storage, transfer and central image reading; nodal specimen harvesting, ex vivo MRX measurements, participant's clinical care pathology, research specimen transfer, research specimen pathology and central pathology reading.

Postdose evaluations for safety and imaging (including MRIs and biopsies) occurred at Visit 2 (Day 1) and Visit 3-5 (18 hours to 78 hours post-dose). 2 postdose MRI imaging scans were performed, one at ˜24 hours and the second at ˜72 hours. Additional safety evaluations occurred at Visit 6 (Day 7±2 days), End-of-Study Visit (Day 28±3 days).

Assessment of results. While not intending to be bound by any specific theory of operation, contrast agents for MRI are detected indirectly through their ability to perturb water proton relaxation and modify MRI signal intensity. Imaging agent causes changes in the T1 (spin lattice or longitudinal relaxation) & T2 (spin-spin or transverse relaxation) or the T2* (dephasing spin-spin) properties of the local tissue being imaged resulting in image contrast. The core is designed for high magnetic relaxivity resulting in change in T2 contrast. For normal lymph nodes, the imaging agent is taken up by resident macrophages resulting in relatively uniform T2 hypointense (dark) contrast. When tumor cells have metastasized to a node, they supplant the macrophages (either in whole or in part) and, as a result, the homogenous hypointensity normally seen is absent in those areas of the node where tumor cells are present. However, because the imaging agent contains a molecularly targeted nanoparticle (unlike the particles discussed in the literature), specific binding between the target on the tumor and the ligand on the imaging agent according to embodiments herein was found to result in heterogeneous hypointensity, where the nanoparticles have become bound to the tumor cells in the invaded nodes. Untargeted particles show no change in intensity for tumor involved nodes when comparing predose to postdose MRI images, however the presently described solutions has been found to show a change in signal intensity between predose and postdose MRI images for both tumor involved and normal nodes. However, the amount of change in signal intensity for tumor involved nodes is discernably different and distinctive versus the change for normal nodes, which allows for the discrimination of the tumor involved nodes from the normal nodes on the postdose MRI images.

MRI measurements were conducted using a 1.5 T or 3 T clinical scanner. The MRI exam involved placing the participant in supine position on a spine coil, off-centered so that the breast diagnosed with the cancer is placed closer to the center of the magnet. A high-density body array coil is placed over the axilla region of the breast to be imaged. All image acquisitions are performed in the axial orientation. T1, T2, and T2* imaging sequences are used.

It was anticipated that the principal effect of imaging agent will be on T2 and T2* relaxation and hence on T2/T2*-weighted sequences. In addition, contrast enhancement is also expected on T1-weighted images. All sequences except the T2* sequence is acquired with clear breath hold instructions for the participant. The approximate imaging time for the above MR imaging sequences is about 20 minutes. Imaging scanner and protocol are expected to be similar for pre and postdose acquisitions to ensure consistency. All images are transferred to a central radiology lab either through secure file transfer or secure shipping of CDs containing imaging data.

The Central Imaging lab performed a review of the MRI scans in cohorts of six participants. Nodes were assessed by both conventional radiological measures such as size and morphology for predose images and for changes in contrast intensity between predose and postdose MRI scans and the discriminating factors such as homogeneous vs heterogeneous hypointensity patterns for postdose images. The radiologists used these image features to score nodes as “suspicious” or “normal” or “indeterminate” both predose and postdose scans.

Tissues from the lymph node imaged with the imaging agent were collected as formalin fixed specimens. Whenever possible, MRX measurements were performed prior to processing the tissue for pathology.

MRX Results. MRX measurements were conducted ex vivo at a MRX laboratory using a preclinical instrument to determine if the MRX signal was detectable in participant nodes and to inform future clinical instrument parameters. In one participant, samples (3 nodes sliced as 9 specimens) significant MRX signal (3-10× of LOQ) were measured in 8 of 9 specimens (LOQ ˜2.5 μg of iron). Core biopsy specimens did not result in measurable MRX signals. Core biopsy represents 2% to 5% of a full node and is of insufficient size to inform MRX sensitivity for the clinical in vivo use case.

Histopathology was evaluated using hematoxylin & Eosin (H&E), HER-2 and Prussian Blue (iron) stains. Five participants had specimens available for pathology staining. Four participants showed Prussian Blue stain in the lymph nodes confirming presence of iron particles. One participant's specimens had no detectable levels of iron. The same participant did not show any evidence of imaging agent in post-MR images. Either issues with lymphatic drainage or technical issues with injection are suspected. Four participants showed HER-2-positive nodal metastasis, and one participant was negative for tumor.

Final results. Four (4) of six (6) participants were evaluable for MRI vs. pathology concordance at per-participant level. In 3 participants, postdose MRI assessments by central radiologists were in concordance with pathological confirmation of nodal metastasis. Radiologists reported a suspicious node in one participant (pre and postdose) who was pathology negative. Of the 2 non evaluable participants, one participant did not have a pathology specimen and another participant did not show any signs of particle drainage into nodes.

Therefore, the imaging agent disclosed herein is present in lymph nodes after administration by injection in subareolar region. Histopathological examination of excised lymph nodal tissue confirms the presence of tumor cells and the imaging agent in the nodes. Comparison of predose versus postdose MR images discriminates suspicious nodes from the normal nodes as seen by the different postdose intensity patterns as one would expect for nonspecific uptake of imaging agent in normal nodes versus specific binding between the HER-2 targeted imaging agent and HER-2 receptor in tumor containing nodes. These data suggest that combining standard morphological assessments (size and shape) with observable changes in MRI contrast using the nanoparticle solutions described herein improve radiological evaluation, which improves routine axillary clinical assessments and treatment options.

### MRI Imaging Using the Imaging Agent—Second Cohort

The imaging agent was utilized in in vivo MRI studies in an additional seven (7) patients as follows.

Human participant selection was based on the following criteria: participants were diagnosed with HER-2-positive primary breast cancer, who were scheduled to undergo preoperative clinical assessment of the axillary nodal disease followed by core biopsy or pathological confirmation. Participants with node-suspicious by routine axillary assessments took part in this study. Exclusion criteria were known inflammatory breast cancer and prior surgical axillary procedure including sentinel lymph node biopsy (SLNB) or axillary lymph node dissection (ALND) or previous radiation on the ipsilateral side of the breast cancer primary. Participants, who in the judgment of the Investigator, have a likelihood of lymph node metastasis and are either scheduled for sentinel lymph node biopsy (SLNB) and/or axillary lymph node dissection (ALND) (Arm 1) or were scheduled for biopsy of suspicious lymph nodes (Arm 2), or have already had a biopsy of suspicious lymph nodes (Arm 3), were selected for enrolment.

All eligible participants received a single dose of 22.5 mg Fe equivalent of drug substance into the subareolar interstitial tissue or area near and around the primary tumor on Study Day 1. Baseline MRI assessments were performed within 3 days before imaging agent administration (the imaging agent being the biofunctional magnetic nanoparticle solution described herein), and a second MRI was performed at an additional timepoint 18 hours to 78 hours, post-imaging agent administration and before any neoadjuvant therapy. An MR compatible clip insertion is introduced under ultrasound guidance prior to imaging. Either whole lymph node(s) or lymph node tissue from a core biopsy of the clipped lymph node were obtained for histopathology assessment. Specimens from patients 7 and 8 were also used for ex vivo MRX measurements.

The study consisted of a screening, baseline, imaging, and follow-up period. Participants undertook a Screening Visit between Day −28 and Day −1 to determine eligibility in the study. Each participant was dosed in the baseline period or Day 1 of the imaging period after obtaining baseline MRI scan. In each patient, a postdose MRI scan and pathology specimens were obtained in the imaging period. Pathology specimens were obtained from the clip inserted node under ultrasound guidance. Data acquisition and assessments include safety assessments and safety labs; image acquisition, storage, transfer and central image reading; nodal specimen harvesting, ex vivo MRX measurements, participant's clinical care pathology, research specimen transfer, research specimen pathology and central pathology reading.

Postdose evaluations for safety and imaging (including MRIs and biopsies) occurred at Visit 2 (Day 1) and Visit 3 (18 hours to 72 hours postdose). 1 postdose MRI imaging scan was performed, within 18-72 hours time window after imaging agent administration. Additional safety evaluations occurred at Visit 4 (Day 7±2 days), End-of-Study Visit (Day 28±3 days), and a Follow-up telecon (Day 90±14 days).

Assessment of results. In some nodes, the homogenous hypointensity normally seen in entirely normal nodes is present in part of the node, with the rest of the node having heterogenous hypointensity. It is contemplated (or hypothesized) that areas of homogenous hypointensity are of normal nodal tissue with nanoparticle drainage or nonspecific uptake. Areas of heterogenous hypointensity (speckled) are indicative of ligand binding to tumorous cells. Areas of unchanged appearance post contrast indicate non-drainage of nanoparticles. Areas of irregular darkening within a node while the rest of the node is of unchanged appearance or homogenous hypointensity indicate heterogeneous architecture arising from a combination of nonspecific uptake in large normal areas of the node, specific binding with accessible tumor cells and non-drainage areas within a node. It is hypothesized that the route of administration of the nanoparticles being via lymphatic drainage follows the same route as tumor invasion, thereby enabling a postdose appearance of heterogeneous architecture of partial irregular darkening with or without speckled pattern which includes nonspecific uptake by macrophages in areas of node not invaded by tumor vs. specific binding of particles to the tumor in areas of tumor or interface or access to tumor. These differences in particle uptake within the same node indicate the presence of normal nodal tissue and abnormal nodal tissue, thereby indicating suspicious nodes invaded by tumors. Some nodes do not show any particle uptake, presumably due to high tumor load causing blockage of lymphatics duct leading to them. However, the presence of hypointensity in the surrounding adjacent nodes are used to assess nodal status at patient level.

Final results. Four (4) of seven (7) participants were evaluable for MRI vs. pathology concordance at per-node level. In 4 participants, postdose MRI assessments by central radiologists of the clipped and biopsied node were in concordance with pathological confirmation of nodal metastasis in the core biopsy specimen from the same node. Of the 3 non evaluable participants, 2 participants did not show any signs of particle drainage from the injection site, 1 participant had uninterpretable image due to MRI susceptibility artifacts.

FIG. 8 illustrates an example of a post-dose MRI assessment of a node showing heterogenous hypointensity (speckled) appearance. This node was assessed by radiologists as suspicious of having tumors and was confirmed as such by pathology.

FIG. 9 illustrates an example of a post-dose MRI assessment of a node showing heterogenous architecture (partial irregular darkening). This node was assessed by radiologists as suspicious of having tumors and was confirmed as such by pathology.

FIG. 10 illustrates an example of a postdose MRI assessment of a node showing both heterogenous architecture (partial irregular darkening) and heterogenous hypointensity (speckled). This node was assessed by radiologists as suspicious of having tumors and was confirmed by conventional clinical assessment methods as an abnormally enlarged node highly suspicious for tumor and the patient presenting 15 metastatic nodes by pathology.

FIG. 11 illustrates an example of a postdose MRI assessment of a node with no contrast uptake. The node appearances on MRI scan pre contrast and post contrast are the same. However, an adjacent node appears with homogenous hypointensity post contrast, indicating contrast uptake by this adjacent node. It is likely that tumors had invaded the node completely and no contrast could enter the node, but contrast still drained to an adjacent, normal node, giving it a homogenous hypointensity appearance. In this patient, conventional clinical assessments showed 3 enlarged nodes highly suspicious for tumor and one of them were biopsied and pathology positive.

Therefore, the imaging agent disclosed herein is present in lymph nodes after administration by injection in subareolar or peritumoral region. Histopathological examination of excised lymph nodal tissue confirms the presence of tumor cells and the imaging agent in the nodes. Comparison of predose versus postdose MR images discriminates suspicious nodes from the normal nodes as seen by the different postdose intensity patterns as one would expect for nonspecific uptake of imaging agent in normal nodes versus specific binding between the HER2 targeted imaging agent and HER2 receptor in tumor containing nodes.

Other features and advantages of the invention will be apparent from the following detailed description, and from the claims.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

### REFERENCES

Beenken S W, Urist M M, Zhang Y, et al. Axillary lymph node status, but not tumor size, predicts locoregional recurrence and overall survival after mastectomy for breast cancer. Ann Surg 2003;237(5): 732-738; discussion 738-739
Choi H Y, Park M, Seo M, Song E, Shin S Y, Sohn Y M. Preoperative Axillary Lymph Node Evaluation in Breast Cancer: Current Issues and Literature Review. Ultrasound Q (2017) 33(1): 6-14.
H. S. Cody III, N. Houssami, Axillary management in breast cancer: What's new for 2012? The Breast 21, 2012, 411e415412
Giuliano A E, Hunt K K, Ballman K V, et al. Axillary dissection vs no axillary dissection in women with invasive breast cancer and sentinel node metastasis: a randomized clinical trial. JAMA 2011;305(6): 569-575
Giuliano A E, Ballman K V, McCall L, et al. Effect of Axillary Dissection vs No Axillary Dissection on 10-Year Overall Survival Among Women with Invasive Breast Cancer and Sentinel Node Metastasis: The ACOSOG Z0011 (Alliance) Randomized Clinical Trial. JAMA 2017;318(10): 918-926

Jatoi I, Kunkler I H. Omission of sentinel node biopsy for breast cancer: Historical context and future perspectives on a modern controversy', Cancer, 2021, vol. 127, no. 23, pp. 4376-4383.
Leenders M, Kramer G, Belghazi K, et al. Can We Identify or Exclude Extensive Axillary Nodal Involvement in Breast Cancer Patients Preoperatively, J Onc., 2019, doi.org/10.1155/2019/8404035
NCCN Guidelines for Breast Cancer 2022, Version 4.
Reiner T, Engel J, Schmidt M, Offerson B V, Smidt M I, Gentilini O D. Is axillary sentinel lymph node biopsy required in patients who undergo primary breast surgery? Breast Care (Basel), 2018; 13:324-330

Saksena M, Jimenez R, Coopey S, et al. Axillary Ultrasound Evaluation in Breast Cancer Patients: A Multidisciplinary Viewpoint and Middle Ground; Journal of Breast Imaging, 2021, 672-675.

We claim:

1. A method of evaluating a target tissue by magnetic resonance imaging, comprising:
introducing a biofunctional magnetic nanoparticle solution into a subject, wherein the biofunctional magnetic nanoparticle solution is comprised of a plurality of biofunctional magnetic nanoparticle structures, each nanoparticle structure comprises an iron core coated with oleic acid, one or more lipid or polymer, one or more stealth generating compound, and one or more targeting ligand adapted to bind a target molecule;
permitting the biofunctional magnetic nanoparticle solution to bind the target molecule, if present, wherein the target molecule is indicative of a health condition in the subject;
performing magnetic resonance imaging of the subject to obtain a magnetic resonance image or image file representative of a target tissue of the subject; and
assessing the magnetic resonance image or image file for an area of heterogenous hypointensity in the target tissue representative of the presence of biofunctional magnetic nanoparticles bound to the target molecule, whereby identifying the area of heterogenous hypointensity in the target tissue is indicative of the presence of the target molecule in the subject tissue.

2. The method of claim 1, wherein each of the nanoparticle structures within the nanoparticle solution has a diameter of between 10 and 150 nanometers.

3. The method of claim 2, wherein within the biofunctional magnetic nanoparticle solution the diameter of each of the nanoparticle structures in the nanoparticle solution is uniform.

4. The method of claim 2, wherein the biofunctional magnetic nanoparticle solution is an isotonic solution that supports product stability and safe human injection.

5. The method of claim 1, wherein the at least one stealth generating compound is polyethylene glycol polymer.

6. The method of claim 1, wherein the organic coating comprises an organic acid and the polymer coating comprises dextran.

7. The method of claim 1, wherein the targeting ligand is an anti-HER-2 antibody or a functional binding fragment thereof.

8. The method of claim 1, wherein the targeting ligand is trastuzumab or a functional binding fragment thereof.

9. The method of claim 1, wherein one or more lymph nodes are assessed.

10. The method of claim 1, wherein the performing magnetic resonance imaging uses T1 imaging sequence or T2 imaging sequence.

11. The method of claim 1, wherein the biofunctional magnetic nanoparticle solution is injected into a peritumoral region of a tumor present on the human subject.

12. The method of claim 1, wherein identification of heterogenous hypointensity in the target area is further indicative of malignancy of the target tissue.

13. The method of claim 1, further comprising assessing the image or image file of the target tissue for heterogenous architecture.

14. The method of claim 13, wherein identification of heterogenous architecture in the target area is further indicative of malignancy of the target tissue.

15. The method of claim 13, further comprising assessing the morphology of the at target tissue for presence of suspicious tumors.

16. The method of claim 1, further comprising performing magnetic resonance imaging on the subject 24 hours after the first imaging and reevaluating the health condition in the subject.

17. The method of claim 1, wherein the one or more target ligand is capable of specifically binding a protein or cell associated with a cancer.

18. The method of claim 1, wherein the target molecule is an HER2 protein, or domain or region thereof.

19. A method to assess axillary nodal diseases by magnetic resonance imaging, the method comprises:
introducing a biofunctional magnetic nanoparticle solution into a human subject diagnosed with or suspected of having breast cancer;
performing magnetic resonance imaging of the region of interest of the human subject, the region comprising at least one lymph node;
assessing an image of the at least one lymph node for heterogenous hypo-intensity or heterogenous architecture; and
assessing axillary nodal disease of the at least one lymph node,
wherein the biofunctional magnetic nanoparticle solution comprises nanoparticle structures, each structure comprising an iron core surrounded with a layer of organic coating and a layer of polymer coating in contact with the organic coating, a targeting ligand specific for a target molecule conjugated to the polymer coating layer, and at least one stealth generating compound bound to the polymer coating layer, the presence of the target molecule being indicative of the presence of a cancer cell or protein associated with the presence of cancer.

20. A medicament for use in magnetic resonance imaging formulated for use in a method for evaluating and/or treating a health condition characterized by the presence a protein or cell associated with a cancer, the method comprising:
introducing a biofunctional magnetic nanoparticle solution into a human subject diagnosed with or suspected of having breast cancer;
performing magnetic resonance imaging of the region of interest of the human subject, the region comprising at least one lymph node;
assessing an image of the at least one lymph node for heterogenous hypo-intensity or heterogenous architecture; and
assessing axillary nodal disease of the at least one lymph node,
wherein the biofunctional magnetic nanoparticle solution comprises nanoparticle structures, each structure comprising an iron core surrounded with a layer of organic coating and a layer of polymer coating in contact with the organic coating, a targeting ligand specific for a target molecule conjugated to the polymer coating layer, and at least one stealth generating compound bound to the polymer coating layer, the presence of the target molecule being indicative of the presence of a cancer cell or protein associated with the presence of cancer,
wherein identification of heterogenous hypointensity or heterogenous architecture in the target tissue or lymph node is further indicative of malignancy of the subject tissue.
